(19) United States
(12) Patent Application Publication
(10) Pub. No.: US 2005/0203419 A1
(43) Pub. Date: Sep. 15, 2005

(54) Side-firing probe for performing optical spectroscopy during core needle biopsy

(76) Inventors: Nirmala Ramanujam, Janesville, WI (US); Changfang Zhu, Madison, WI (US); Carmalyn Lubawy, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/063,273

(22) Filed: Feb. 22, 2005

Related U.S. Application Data
(60) Provisional application No. 60/547,262, filed on Feb. 24, 2004.
(60) Provisional application No. 60/553,825, filed on Mar. 17, 2004.
(60) Provisional application No. 60/615,671, filed on Oct. 4, 2004.

(51) Int. Cl. A61B 6/00

(52) U.S. Cl. 600/473

(57) ABSTRACT

A needle biopsy includes the step of inserting an optical spectroscopy probe in the needle and gathering optical information through a window formed in the side of the needle at its distal end. The optical probe includes an illumination optical fiber which conveys light to the tissues adjacent the side window and a detection optical fiber which collects light from the same tissues and conveys it to an optical spectroscopy instrument. Based on the results of the optical spectroscopy measurement, the optical probe may be withdrawn from the needle and a cutter advanced to acquire a sample of the tissues adjacent the side window.

### CROSS-REFERENCE TO RELATED APPLICATIONS

This application claims the benefit of U.S. Provisional patent application Ser. No. 60/547,262 filed on Feb. 24, 2004 and entitled “SIDE-FIRING OPTICAL PROBE FOR CORE NEEDLE BIOPSY”; U.S. Provisional patent application Ser. No. 60/553,825 filed on Mar. 17, 2004 and entitled “ENDOSCOPICALLY COMPATIBLE NEAR INFRARED PHOTON”; and U.S. Provisional patent application Ser. No. 60/615,671 filed on Oct. 4, 2004 and entitled “OPTICAL SENSOR FOR BREAST CANCER DETECTION DURING BIOPSY”.

### STATEMENT REGARDING FEDERALLY SPONSORED RESEARCH

This invention was made with government support under Grant No. NIH EB00184 awarded by the National Institute of Health. The United States Government has certain rights in this invention.

### BACKGROUND OF THE INVENTION

The field of the invention is optical spectroscopy, and particularly, the use of optical spectroscopy during the performance of core needle biopsy procedures.

Percutaneous, image-guided core needle biopsy is being increasingly used to diagnose breast lesions. Compared to surgical biopsy, this procedure is less invasive, less expensive, faster, minimizes deformity, leaves little or no scarring and requires a shorter time for recovery. Needle biopsy can obviate the need for surgery in women with benign lesions and reduce the number of surgical procedures performed in women with breast cancer. However, the caveat is that needle biopsy has a limited sampling accuracy because only a few small pieces of tissue are extracted from random locations in the suspicious mass. In some cases, sampling of the suspicious mass may be missed altogether. Consequences include a false-negative rate of up to 7% (when verified with follow up mammography) and repeat biopsies (percutaneous or surgical) in up to 18% of patients (due to discordance between histological findings and mammography). The sampling accuracy of core needle biopsy is highly dependent on operator skills and on the equipment used.

Optical spectroscopy may be used to characterize tissues. These methods include ultraviolet-visible (UV-VIS) reflectance and fluorescence spectroscopy and Near infrared (NIR) optical spectroscopy.

Near infrared (NIR) optical spectroscopy is a technique in which a light source is placed on the tissue surface launches photon density waves into the tissue having a wavelength in the range of 600 nm to 1000 nm. A fraction of these photons, which propagate through the tissue, reach a collector some distance (0.5 cm to 8 cm) from the light source. The collected photons, on average, have traversed a banana shaped path within the tissues which extends into the tissue a distance equal to approximately half the separation between the source and the collector. The absorption and scattering properties of the tissue can be retrieved from the amplitude and phase shift of the collected light using a light transport algorithm based on the Diffusion equation. The concentrations of absorbers can be derived from the absorption coefficient using Beer's law. Endogenous absorbers in breast tissue at NIR wavelengths include oxy and deoxy hemoglobin, water and lipids. The scattering is associated with microscopic variations in the size, shape and refractive indices of both intracellular and extra cellular components.

Tissue vascularity, hemoglobin concentration and saturation have all been identified as diagnostic markers of breast cancer using a variety of different techniques including immunohistochemistry, needle oxygen electrodes and magnetic resonance spectroscopy. Breast cancers are more vascularized and are hypoxic compared to normal breast tissues. A number of groups have demonstrated that these sources of contrast can be exploited for the non-invasive detection of breast cancer in the intact breast using NIR diffuse optical imaging. For example, Ntziachristos et al developed and tested a novel hybrid system that combines magnetic resonance imaging and NIR diffuse optical imaging for non-invasive detection of breast cancer. Using this technique, they quantified oxygenated hemoglobin (HbO₂) and deoxygenated hemoglobin (Hb) concentrations of five malignant and nine benign breast lesions in vivo. The average total hemoglobin concentration for the cancers, fibroadenomas and normal tissues were 0.130±0.100 mM, 0.060±0.010 mM and 0.018±0.005 mM, respectively. This representative study demonstrates that NIR diffuse optical methods can discriminate malignant from benign lesions based on tissue vascularity.

Ultraviolet-visible (UV-VIS) reflectance and fluorescence spectroscopy (RFS) is a combination of two techniques. Reflectance spectroscopy is a technique in which broad spectrum light containing wavelengths from 350 nm to 600 nm illuminates the tissue. The reflected light is collected, separated into its component wavelengths and measured. This enables us to examine several chemicals which absorb light including oxy and deoxy hemoglobin, and beta-carotene. Fluorescence spectroscopy is a technique where a single wavelength is used to illuminate the tissue. The illumination light is absorbed by endogenous and/or exogenous chemicals in the body, then re-emitted as fluorescence light at a different wavelength. This re-emitted light is collected and measured. This is done for a series of illumination wavelengths of light in the range of 300 to 460 nm. Fluorescence spectroscopy allows us to characterize several tissue components such as FAD, NADH, collagen and Tryptophan. These two techniques can be done in rapid succession, with a single instrument.

All of the optical spectroscopy techniques require that the light source and light detector be positioned close to the tissues to be examined. In both methods the measured properties are averages of all the tissues where the light has traversed. In the former method, small areas inside large tissues can be difficult to distinguish without complex imaging techniques. The UV-VIS light used in the latter method does not penetrate deeply into human tissue and this is typically used to examine the surface of tissues. The light may also be delivered to a tissue through an optical fiber that extends through an endoscope such as that described in U.S. Pat. No. 5,131,398 to examine the surface of an internal organ.

### SUMMARY OF THE INVENTION

The present invention is a method and optical probe for making optical spectroscopy measurements during the performance of a core needle biopsy. The optical probe is inserted into the biopsy needle after the needle has been inserted into the candidate tissue to be biopsied; light is applied to the probe and is emitted into tissue surrounding the tip of the biopsy needle; and light from these tissues is collected by the probe and conveyed to a spectroscopy instrument for analysis. When target tissue is detected, the probe is removed from the biopsy needle and a tissue sample is acquired by advancing a cutting tool.

The optical probe includes: an end cap which is sized and shaped to fit into the central opening of a biopsy needle; one or more illumination fibers having a distal end retained to the probe end cap and a proximal end which connects to a source of light; one or more detector fibers having a distal end retained to the probe end cap and a proximal end connected to a spectroscopic instrument; and wherein the probe end cap may be positioned near the distal end of the needle to emit light from the distal end of the illumination fiber through a window formed in the side of the biopsy needle and receive light from tissues through the window and convey it through the detector fiber to the spectroscopic instrument.

By inserting the optical probe in the biopsy needle and examining the tissue surrounding the tip of the needle, the candidate tissue can be evaluated prior to the biopsy. This enables different and larger regions to be examined before the biopsy is taken, thus increasing the probability that the correct tissue is biopsied and that another biopsy will not be required. This method has the potential to improve the lives of thousands of women by eliminating the need for 90,000 to 180,000 repeat biopsies per year and improving the accuracy of diagnosis for thousands more. This will significantly alleviate the physical and emotional costs to thousands of women undergoing this procedure.

A general object of the invention is to provide an optical spectroscopy system which improves the sampling accuracy of an image guided core needle biopsy. The system includes optical fibers that are inserted into the bore of a biopsy needle to illuminate and acquire light from surrounding tissues, and a spectrometer which receives this light and provides a measure of tissue physiological parameters. Parameters such as tissue vascularity and fluorescent spectra distinguish malignant from non-malignant breast tissue.

### BRIEF DESCRIPTION OF THE DRAWINGS

FIG. 1 is a pictorial view of an optical spectrometer, side firing probe and needle which employs the present invention;

FIG. 2 is a schematic representation of an optical probe in a biopsy needle according to the present invention;

FIG. 3 is a pictorial view of a preferred embodiment of a NIR optical probe in a biopsy needle;

FIG. 4 is a pictorial view of the probe and needle of FIG. 3 in cross-section;

FIG. 5 is a schematic diagram of a NIR optical spectroscopy instrument used with the optical probe of FIG. 3;

FIG. 6 is a pictorial view of a preferred embodiment of a UV-VIS spectroscopy optical probe in a biopsy needle;

FIG. 7 is an alternative embodiment of an optical spectroscopy probe; and

FIG. 8 is a view in cross-section of the needle with a cutter deployed in place of the optical probe.

### DETAILED DESCRIPTION OF THE PREFERRED EMBODIMENT

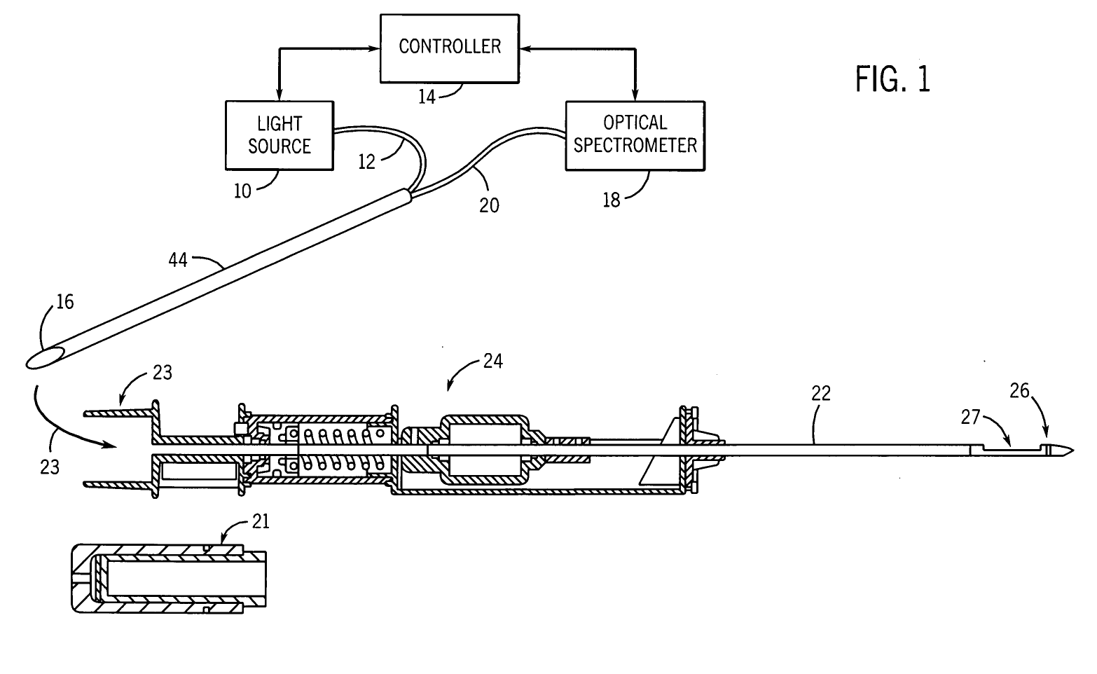

Referring particularly to FIG. 1, an optical spectroscopy system which employs the present invention includes a light source 10 that connects to the proximal end of an illumination optical fiber 12 and is operated by a controller 14 to produce light of the proper wavelength at a probe 16. The controller 14 also operates an optical spectrometer instrument 18 which receives light through a detector optical fiber 20 which has a distal end fastened to the probe 16. The diameter of the probe 16 is sufficiently small that it can be inserted into a central channel formed in a biopsy needle shank 22. A breast biopsy needle such as that disclosed in U.S. Pat. No. 6,638,235 and manufactured by Suros Surgical Systems, Inc. is employed in a preferred embodiment. The needle shank 22 extends completely through a handle 24 that contains a mechanism for extracting a tissue sample through the needle 22. For a 9-gauge needle, the shank 22 has a length of 12 cm and a 3.7 mm outside diameter. A sample holder 21 attached to a connector 23 on the proximal end of the handle 24 is removed during the optical data acquisition portion of the procedure to provide access to the proximal end of the needle shank 22 for optical probe 16. When this step of the procedure is completed, the optical probe 16 is withdrawn and the sample holder 21 is reattached.

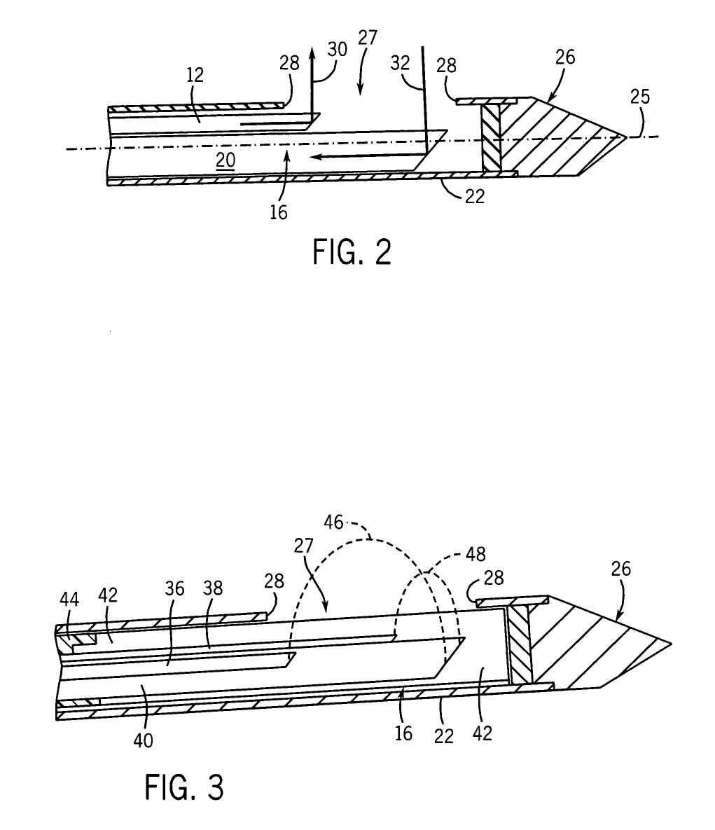

As shown in FIG. 2, when the optical probe 16 is properly positioned at the distal end 26 of the needle shank 22 the distal ends of both optical fibers 12 and 20 are aligned axially along a longitudinal axis 25 with a window 27 formed by an opening 28 in the shank 22 of the needle near its distal end 26. This window is 20 mm long and 3.7 mm wide in the preferred needle and is used during the biopsy procedure for tissue collection. The distal end of illumination optical fiber 12 is cut at a 43° angle such that light emanating from light source 10 and traveling through the fiber 12 is reflected radially outward through the window 27 into surrounding tissue. That is, the illumination light travels axially along the length of the biopsy needle shank 22 and is redirected by the bevelled distal end to make a substantially right angle turn as indicated by arrow 30.

The distal end of the detection optical fiber 20 is similarly beveled at a 43° angle. As a result, light entering through the window 27 in a substantially radially inward direction is reflected off the beveled end and is redirected axially along the optical fiber 20 as indicated by arrow 32. Illumination of tissues located in the region outside the window 27 is thus performed by conveying the desired light along fiber 12 and collecting the resulting light produced in these tissues with the optical fiber 20. The collected light is conveyed back to the optical spectrometer 18 by the detector optical fiber 20. Tissues surrounding the distal tip 26 of the biopsy needle 22 can thus be spectroscopically examined by rotating the needle 22 about the longitudinal axis 25 to “aim” the window 27 in a succession of radial directions.

As will now be described in more detail, the number and size of the optical fibers as well as their positioning in the optical probe 16 will depend upon the particular spectroscopic measurement being made. It is contemplated that a number of different probes 16 may be used in any single biopsy procedure in order to gather enough information to make a clinical decision. The biopsy needle 22 is inserted in the patient and its distal end 26 is guided to the candidate tissues using an imaging modality such as ultrasound or MRI. An optical probe 16 may then be inserted into the needle 22 and oriented as described above to acquire optical information for the spectrometer 18. This may be repeated using the same or a different probe 16 until a decision is made to biopsy. The optical probe 16 is then withdrawn from the needle shank 22. A gentle vacuum is applied to the needle, pulling a small amount of tissue in the window 27. A cutter 29 is then advanced, as shown in FIG. 8, slicing this tissue where it enters the needle. The vacuum then pulls this sample of tissue down the needle's length and into a collection chamber. It can be appreciated that by performing optical spectroscopy through window 27 on the very same tissue that is removed by the biopsy step, highly reliable clinical information is acquired.

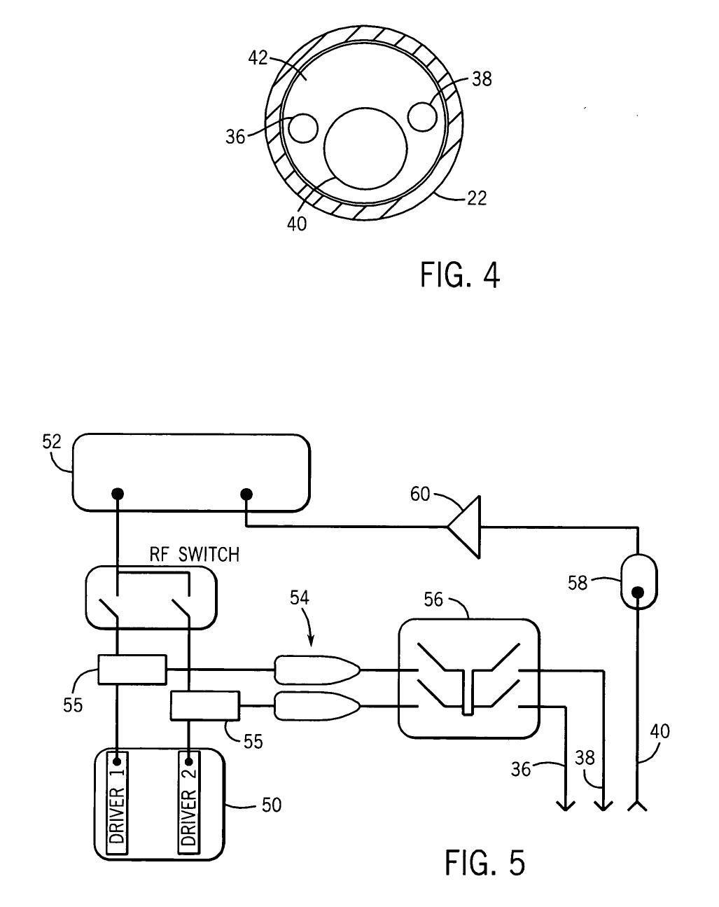

Referring particularly to FIGS. 3 and 4, the first preferred embodiment of the invention is a photon migration spectroscopy technique in which near infrared (NIR) photon density waves are launched into the tissue using two illumination optical fibers 36 and 38 and reflected light is gathered from the tissue using a single detection optical fiber 40. The distal ends of these optical fibers 36, 38 and 40 are encased in a rigid, optically transparent quartz end cap 42 to form the optical probe 16. Flexible tubing 44 such as that sold under the trademark “TYGON” fastens to the cap 42 and extends along the length of the optical fibers to hold them together and provide a limited amount of protection. At the proximal end of this Tygon tubing there is an aluminum junction (not shown), which bifurcates into three parts, one for each fiber. Each of these parts is sheathed in polyvinyl chloride (PVC). After the junction all of the fibers are reinforced with Kevlar fibers and each fiber is terminated in a “Fiber Connector”, which connects the sensor to the NIR instrument described below.

The optical fiber diameters and the spacing between their distal ends are selected to optimize the signal level and the depth of the tissue that can be measured outside the window 27. In the resulting optical probe 16 the two illumination optical fibers 36 and 38 have a diameter of 200 μm and a numerical aperture of 0.22; the detection optical fiber 40 has a core diameter of 600 μm and a numerical aperture of 0.22; and quartz end cap 42 has a length of 25 mm and an outer diameter of 2.4 mm. All optical fiber distal ends are polished at an angle of 43° and radially oriented such that the light from each fiber is normal to the cylindrical surface of the quartz end cap 42. This radial orientation minimizes specular reflection into the collection fiber 40.

The relative placement and orientation of the fiber tips is fixed by gluing the fibers 36, 38 and 40 together. Epoxy at the junction of the end cap 42 and the flexible tube 44 fixes the fibers inside the quartz end cap 42. The outer diameter of the cap 42 is stepped down at its proximal end so it fits inside the distal end of flexible tube 44. This provides strength at the junction and makes the junction smooth for easy insertion and removal of the probe 16 from the biopsy needle 22. The distal end of illumination fiber 36 is spaced 10 mm from the distal end of detector fiber 40 to provide deeper penetration and probe deeper into the tissues as indicated by dashed line 46. The tip of the other illumination fiber 38 is spaced only 5 mm from the detector fiber tip to probe at a much shallower depth as indicate by dashed line 48. This latter depth approximates the depth of tissue acquired by the subsequent biopsy. The preferred probe 16 thus enables two measurements to be made at different spacings. This enables automatic correction for instrument response and collection fiber bending loss, thus obviating the need for the use of calibration phantoms.

Referring particularly to FIG. 5, the instrument to which this NIR probe is coupled, is a frequency domain system similar to that described by T H Pham, P Coquoz, J B Fishkin, E Anderson, B J Troberg, in a publication entitled “Broad Bandwidth Frequency Domain Instrument For Quantitative Tissue Optical Spectroscopy,” Review Of Scientific Instruments 71, 2500-2513 (2000). It consists of a laser diode driver 50 (ILX lightwave LDC-3908) and a network analyzer 52 (Agilent 9712ET), which amplitude modulates 811 nm and 850 nm laser diodes 54 (JDS Uniphase SDL5421-H1-810, JDS Uniphase SDL 5401-G1-852). Associated with each laser diode 54 is a bias-tee 55 for combining direct current bias and rf power. The output of the laser 54 is connected to the illumination optical fibers 36 and 38 through an optical switch 56 (Dicon GP700). The two laser diode wavelengths lie within the absorption bands of HB and H₂0. The average power delivered by lasers 54 at the probe tip is approximately 6 mW.

The probe's collection fiber 40 is connected to an avalanche photo diode 58 (Hamamatsu C5658), and a 19 dB amplifier 60 (Mini Circuits ZFC-500HLN). For each measurement, 101 data points are collected over the frequency range of 50-150 MHz. At a bandwidth of 15 Hz (resulting in a measurement sweep time of 8 seconds), the signal to noise ratio of the instrument and probe is greater than 300:1 over the 50 to 150 MHz range in a liquid phantom with an absorption coefficient of 0.2 cm⁻¹ and reduced scattering coefficient of 10 cm⁻¹. This instrument records phase and amplitude data. This data is fit to an infinite solution of the diffusion equation, which is appropriate for the interstitial geometry used in the breast needle biopsy application.

The present invention may also be used to perform ultraviolet-visible reflectance and fluorescence spectroscopy. Because light at these wavelengths does not penetrate deeply into human tissue, the distal ends of the illumination fiber and the detection fiber must be spaced much closer together than the 5 mm to 10 mm range used in the NIR embodiment described above.

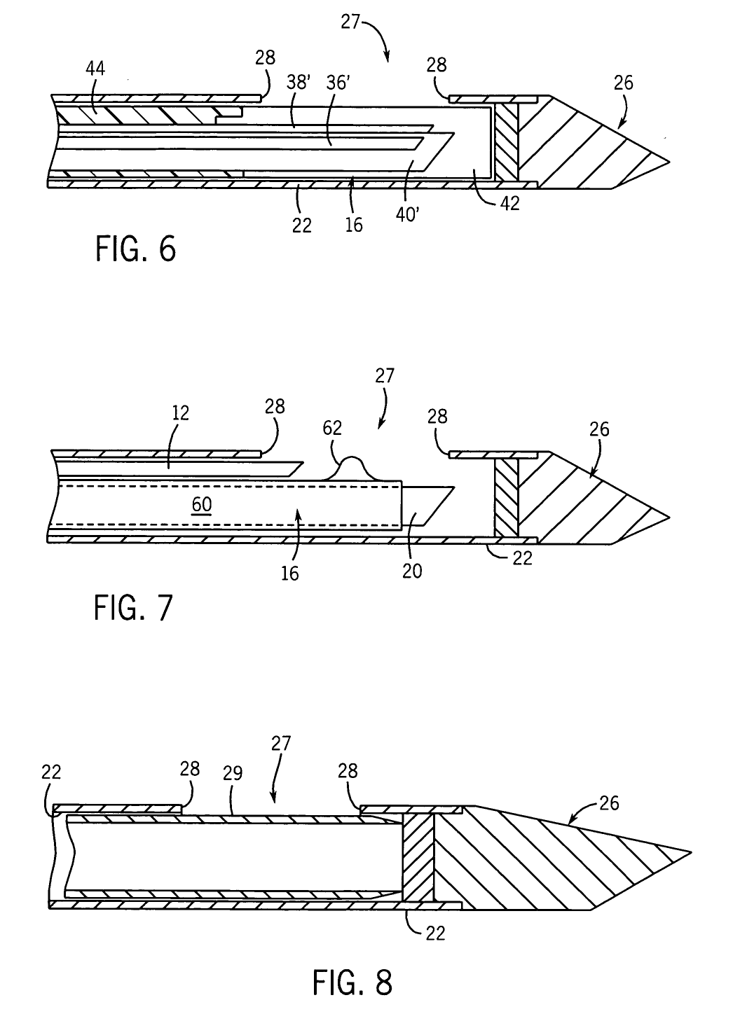

Referring particularly to FIG. 6, a preferred embodiment of an optical probe 16 for use at ultraviolet and visible wavelengths is substantially the same as that described above for NIR applications. In this case, however, the larger 600 μm optical fiber 40′ is employed to illuminate tissue and the smaller 200 μm optical fibers 36′ and 38′ are employed to detect light received from the tissue. The optical fibers 36′, 38′ and 40′ are bonded together and sealed inside the quartz end cap 42 as described above, however, the spacing between their distal ends is significantly smaller. The distal ends of collection fibers 36′ and 38′ are positioned near the distal end of illumination fiber 40′, but they are disposed on opposite sides of the illumination fiber 40′ and their bevelled tips are oriented at an angle such that their collection regions are disposed circumferentially around the quartz end cap 42, but do not overlap each other. The use of two collection fibers thus increases the field of view of the probe.

The instrument to which this optical probe is connected is significantly different than the NIR instrument described above. In this embodiment the light source 10 is a broad band light source with a monochromator coupled to its output to select a single wavelength of light for application to the illumination fiber 40′. The light collected by detector fibers 36′ and 38′ is coupled to a grating to separate the light into its component colors. The separated light is applied to a CCD device which measures the amplitude of each component color.

In some clinical applications it is desirable to reduce the “leakage” of light that can occur between the illumination and detector optical fibers. An alternative embodiment of the invention which minimizes such leakage is shown in FIG. 7. This is a variation of the embodiment described above with respect to FIG. 2 in which like elements are indicated with the same reference numbers. The difference in this alternative embodiment is that an opaque sheath 60 is disposed around the detection optical fiber 20 to block any leakage of light into the optical fiber 20 along its length. The selection of material will depend on the wavelength of light to be blocked. In addition, a light baffle 62 is disposed between the distal end of the illuminating optical fiber 12 and the distal end of the detector optical fiber 20. Baffle 62 is formed from an opaque material for the wavelengths used and it extends radially outward from the surface of the opaque sheath 60 as far as possible without interference with the quartz end cap 42. It extends circumferentially around the opaque sheath 60 a sufficient distance to serve as a light barrier between the optical fiber tips. In this embodiment a higher percentage of the light produced by illuminating optical fiber 12 passes through the subject tissues before being captured by the detection optical fiber 20.

It should be apparent that variations from the preferred embodiment described above are possible without departing from the spirit of the present invention. Cutting the ends of the optical fibers at a 43° angle with respect to longitudinal axis 25 reflects the light at approximately 90°. Depending on the size of the window 27 and the location of the distal end of an optical fiber, other angles are possible. Also, mirror like structures such as gold can be added as a coating to the bevelled tips of the optical fibers to increase the percentage of light that is reflected to and from the tissues.

1. A method for performing a needle biopsy, the steps comprising:
a) inserting the needle into the tissues to be biopsied with an aperture in the side of the needle located near target tissues;
b) inserting an optical probe into the biopsy needle
c) illuminating the target tissue with light directed through one optical fiber in the optical probe and out the side aperture;
d) collecting light emitted by the target tissue by capturing light entering the optical probe through the side aperture with a second optical fiber;
e) conveying through the second optical fiber the collected light to a spectroscopy instrument for processing;
f) removing the optical probe from the biopsy needle; and
g) advancing a biopsy cutting tool for acquiring a sample of target tissues.

2. An apparatus for acquiring optical data during a needle biopsy, the combination comprising;
a plurality of optical fibers formed as a probe having a diameter sufficiently small to be inserted into the needle at its proximal end and having a length sufficient to extend along the entire longitudinal extent of the needle and position the distal end of the optical fibers adjacent a side window located near the distal end of the needle;
means for connecting the proximal ends of the optical fibers to a spectroscopy instrument;
means formed at the distal end of each optical fiber for reflecting light substantially perpendicular to the longitudinal axis of the needle, such that light produced by the spectroscopy instrument travels through an illumination optical fiber and is reflected out its distal end through the side window formed in the distal end of the needle, and light entering through the side window enters the distal end of detector optical fiber and is conveyed by the detector optical fiber back to the spectroscopy instrument.

3. The apparatus as recited in claim 2 in which the means formed at the distal end of each optical fiber is a reflected surface formed by cutting the distal end of the optical fiber at an angle.

4. The apparatus as recited in claim 3 in which the angle is substantially 45° with respect to the longitudinal axis.

5. The apparatus as recited in claim 3 in which the angled ends of the optical fibers are coated with a reflective material.

6. The apparatus as recited in claim 2 in which the distal ends of the optical fibers are enclosed in an end cap formed with a transparent material.

7. The apparatus as recited in claim 6 in which the transparent material is quartz.

8. The apparatus as recited in claim 2 in which the spectroscopy instrument is a near infrared optical spectroscopy instrument.

9. The apparatus as recited in claim 2 in which the light produced by the spectroscopy instrument has a wavelength in the ultra violet to visible range.

10. The apparatus as recited in claim 2 in which the probe includes two illumination optical fibers connected at their proximal ends to the spectrometer instrument and having their distal ends located at two different respective distances from the distal end of the detector optical fiber.

11. An optical probe for insertion into the shank of a biopsy needle to acquire optical data for an optical spectrometer, the combination comprising:
a detector optical fiber having a substantially cylindrical shape with a diameter smaller than a shank of the biopsy needle and a length longer than the biopsy needle;
a connector fastened to the proximal end of the detector optical fiber for attachment to the optical spectrometer;
means on the distal end of the detector optical fiber for receiving light from a radial direction and redirecting the light along the longitudinal axis of the detector optical fiber;
an illuminating optical fiber having a substantially cylindrical shape with a diameter smaller than the shank of the biopsy needle and a length longer than the biopsy needle;
a connector fastened to the proximal end of the illumination optical fiber for attachment to a light source;
means on the distal end of the illumination optical fiber for receiving light conveyed by the illumination optical fiber from an attached light source and redirecting the light radially outward from its longitudinal axis;
means for fastening the distal ends of the illumination optical fiber and the detector optical fiber together such that their radial directions are substantially aligned and their ends are spaced apart a predetermined distance.

12. The optical probe as recited in claim 11 in which the means for redirecting light on each of the distal ends of the optical fibers is a reflective surface formed by cutting the end of the optical fiber at an angle with respect to its longitudinal axis.

13. The optical probe as recited in claim 12 in which the angle is substantially 45°.

14. The optical probe as recited in claim 12 in which a reflective substance is deposited on the distal end of each optical fiber.

15. The optical probe as recited in claim 11 in which an optically opaque light baffle is disposed on the cylindrical outer surface of one optical fiber and positioned between the distal ends of the optical fibers to block light from directly reaching the detector optical fiber from the illumination optical fiber.

16. The optical probe as recited in claim 15 in which an opaque sheath is formed around the distal end of the detector optical fiber and the light baffle is fastened to the opaque sheath.
